(19) United States
(12) Patent Application Publication
(10) Pub. No.: US 2024/0366409 A1
(43) Pub. Date: Nov. 7, 2024

(54) SIZING METHOD AND COMPUTER EQUIPMENT FOR BRAIDED STENTS

(71) Applicant: ArteryFlow Technology Co., Ltd., Hangzhou (CN)

(72) Inventors: Jianping XIANG, Hangzhou (CN); Yejie SHAN, Hangzhou (CN)

(21) Appl. No.: 18/651,476

(22) Filed: Apr. 30, 2024

(30) Foreign Application Priority Data
May 4, 2023 (CN) 2023104877863

(51) Int. Cl. A61F 2/95; A61F 2/07; G16H 50/50

(52) U.S. Cl. CPC A61F 2/95; A61F 2/07; G16H 50/50

(57) ABSTRACT

This application relates to a sizing method and computer equipment for braided stents. The sizing method includes: obtaining geometric models of aneurysm and parent artery, determining the expected landing zone of the braided stent, obtaining the centerline and cross-section of the blood vessel at the expected landing zone; obtaining the proposed diameter of the braided stent, thereby obtaining the first braided stent that meets the expectations, obtaining the anchoring section length of the first braided stent, obtaining the working zone length; discretizing the working zone into a finite number of discrete segments, obtaining the corresponding relationship between the length and diameter of the discrete segments, obtaining the diameter of the discrete segments, obtaining the length of the discrete segments based on the corresponding relationship and accumulating them until the total length equates the working zone length, obtaining the number of discrete segments.

### CROSS-REFERENCE TO RELATED APPLICATION

This application claims priority from the Chinese patent application 2023104877863 filed May 4, 2023, the content of which is incorporated herein in the entirety by reference.

### TECHNICAL FIELD

This application relates to the field of medical engineering technology, specifically to a sizing method and a computer equipment for braided stents.

### BACKGROUND

Intracranial aneurysm refers to the abnormal bulging of the intracranial arterial wall, with an overall incidence rate of approximately 3% to 5%. Although most intracranial aneurysms do not rupture throughout a person's lifetime, once ruptured, leading to subarachnoid hemorrhage, the mortality rate can reach 40%. Therefore, timely screening and intervention for intracranial aneurysms are crucial.

Currently, the interventional treatment for small to medium-sized aneurysms, especially ruptured ones, mainly involves using metallic coils to embolize the aneurysm sac. This process helps slow down the impact of blood flow on the aneurysm wall, leading to the formation of an intrasaccular thrombus and ultimately achieving embolization of the aneurysm sac. For wide-necked or large and fusiform aneurysms, dense mesh braided stents can achieve better therapeutic effects.

After implantation in blood vessels, the metal coverage ratio at the neck of the intracranial aneurysm plays a crucial role in occluding the aneurysm. Conversely, a high metal coverage rate in non-neck areas poses a risk of occluding healthy branches. Therefore, some manufacturers have developed non-uniform dense mesh braided stents to ensure occlusion of the aneurysm sac while reducing the risk of occluding side branches.

Due to the significant foreshortening behavior of dense mesh braided stents, the length after implantation in blood vessels is difficult to accurately predict. The non-uniformity of the stent mesh introduces non-uniform foreshortening behavior, making it even more challenging to predict the length of the stent. As a result, there is a lack of reference for selecting braided stents, increasing the surgical risk and necessitating reliance on experience to assess the effectiveness of dense mesh braided stents in blood vessel implantation.

### SUMMARY

Based on this, it is necessary to provide a sizing method for braided stents to address the aforementioned technical issues.

The sizing method for braided stents includes:
Obtaining geometric models of aneurysm and parent artery, determining the expected landing zone of the braided stent, obtaining the centerline and cross-section of the blood vessel at the expected landing zone, wherein the expected landing zone includes an anchoring zones and a working zone, and the braided stent comprises a working section for occluding the aneurysm and an anchoring section for anchoring;
Based on the centerline and cross-section of the blood vessel in the anchoring zone, obtaining the proposed diameter of the braided stent, thereby obtaining the first braided stent that meets the expectations, obtaining the anchoring section length of the first braided stent, obtaining the working zone length based on the difference between the expected landing zone length and the anchoring section length;
Discretizing the working zone into a finite number of discrete segments, obtaining the corresponding relationship between the length and diameter of the discrete segments, obtaining the diameter of the discrete segments based on the working zone length, the centerline, and the cross-section of the working zone, obtaining the length of the discrete segments based on the corresponding relationship and accumulating them until the total length equates the working zone length, obtaining the number of discrete segments;
Based on the number of discrete segments and the total nominal lengths of the discrete segments in the first braided stent, select a second braided stent whose working section length matches the expectations.

Optionally, the braided stent is a non-uniform braided stent, wherein the anchoring section comprises two parts located at two opposite ends of the working section, and the braiding density of the working section is higher than that of the anchoring section.

Optionally, obtaining the proposed diameter of the braided stent based on the centerline and cross-section of the anchoring zone, specifically comprising:

The anchoring zone includes a distal anchoring zone and a proximal anchoring zone, wherein the distal anchoring zone has a distal anchoring point relatively far from the aneurysm, and the proximal anchoring zone has a proximal anchoring point relatively far from the aneurysm. Selecting the larger radius of the two, either the distal anchoring point or the proximal anchoring point of the blood vessel centerline as the half of reference diameter, and obtaining the proposed diameter based on the reference diameter.

Optionally, discretizing the working zone into a finite number of discrete segments and obtaining the corresponding relationship between the length and diameter of the discrete segments, specifically comprising:

The repeating unit cell of the working section is a rhombus formed by the intersection of the stent wires, with the length of the rhombus along the axial direction of the braided stent being the length of the discrete segment. Based on the relationship between the length and height of the rhombus and the circumferential arrangement of the rhombi, obtain the corresponding relationship between the length and diameter of the discrete segments.

Optionally, discretizing the working zone into a finite number of discrete segments and obtaining the corresponding relationship between the length and diameter of the discrete segments, utilizing the following equation:

$${l}_{a 2} ( D ) = 2 \sqrt{{w}_{2}^{2} - \frac{{π}^{2} {( D - d )}^{2}}{{N}^{2}}}$$

In the equation, lₐ₂(D) represents the length of the discrete segment, D represents the diameter of the discrete segment;
w₂ represents the rhombus side length, N represents the number of stent wires, d represents the diameter of the stent wires, all of which are parameters of the first braided stent.

Optionally, obtaining the diameter of the discrete segments based on the length of the working zone, the centerline, and the cross-section of the working zone, utilizing the following equation:

$${R}^{*} ( s ) = min ( R ( s ) , \frac{1}{2} {D}_{free} )$$

In the equation, s represents a point on the blood vessel centerline, R(s) represents the radius of the blood vessel centerline, R*(s) represents the radius of the discrete segment which is used to obtain the diameter of the discrete segment, and D_{free} represents the upper limit of the expanded diameter of the first braided stent in its natural released state.

Optionally, the discrete segments comprising sequentially determined first and second discrete segments;

Obtaining the length of the discrete segments based on the corresponding relationship, specifically comprising:

Determining the starting point of the first discrete segment in the working zone, obtaining the length of the first discrete segment based on the corresponding relationship;

Using the endpoint of the first discrete segment as the starting point of the second discrete segment, obtaining the length of the second discrete segment based on the corresponding relationship.

Optionally, obtaining the number of discrete segments until the total length equates to the length of the working zone, specifically comprising:

Obtaining the accumulated number of discrete segments when the total length of the discrete segments equates to the length of the working zone.

Optionally, the sizing method for braided stents further includes simulating the implantation of the working section of the second braided stent:

Obtaining the length of the working section of the second braided stent, discretizing the length of the working section of the second braided stent into a finite number of discrete segments, obtaining the diameter of the discrete segments based on the expected landing zone, the centerline, and the cross-section of the blood vessel at the expected landing zone;

Obtaining the length of the discrete segments based on the diameter of the discrete segment and corresponding relationship, simulating the implantation of the working section of the second braided stent into the working zone, until the simulation of implantation of all discrete segments of the second braided stent is completed, and obtaining the endpoint of the working section of the second braided stent after implantation.

The present application also provides a set of computer equipment, which comprises a memory, a processor, and a computer program stored in the memory. The processor executes the computer program to implement the steps of the sizing method of braided stents described in the present application.

This application has at least the following effects:

The anchoring section of this application is used to obtain the proposed diameter of the braided stent, while the working section is used to obtain the proposed length of the braided stent's working section. The proposed diameter is obtained based on the size of the vessel lumen in the anchoring zone. By simulating the implantation status of discrete segments into the working zone, the number of discrete segments required to fill the working zone length under the proposed diameter is obtained, and then a second braided stent that meets the requirement of the working zone length is selected. Through the proposal based on these two dimensions, a braided stent suitable for the patient to be implanted is finally obtained, providing reference basis for the selection of the braided stent and assisting doctors in accurate and efficient planning of the stent model before or during surgery.

### BRIEF DESCRIPTION OF THE DRAWINGS

FIG. 1 depicts the flow chart of sizing method for braided stents in one embodiment of the present application;

FIG. 2 illustrates the schematic diagram of the geometric model of aneurysm and the parent artery in one embodiment of the present application;

FIG. 3 shows the schematic diagram of the geometric structure of a non-uniform braided stent in one embodiment of the present application;

FIG. 4 represents the schematic diagram of a non-uniform braided stent and its discrete segments in one embodiment of the present application;

FIG. 5 displays the internal structure diagram of a computer device in one embodiment.

### DETAILED DESCRIPTION OF THE EXAMPLE EMBODIMENTS

In order to make the purpose, technical solution and advantages of the present application clearer, the present application will be further described in detail below in conjunction with the accompanying drawings and embodiments. It should be understood that the specific embodiments described here are only used to explain the present application, not to limit the present application.

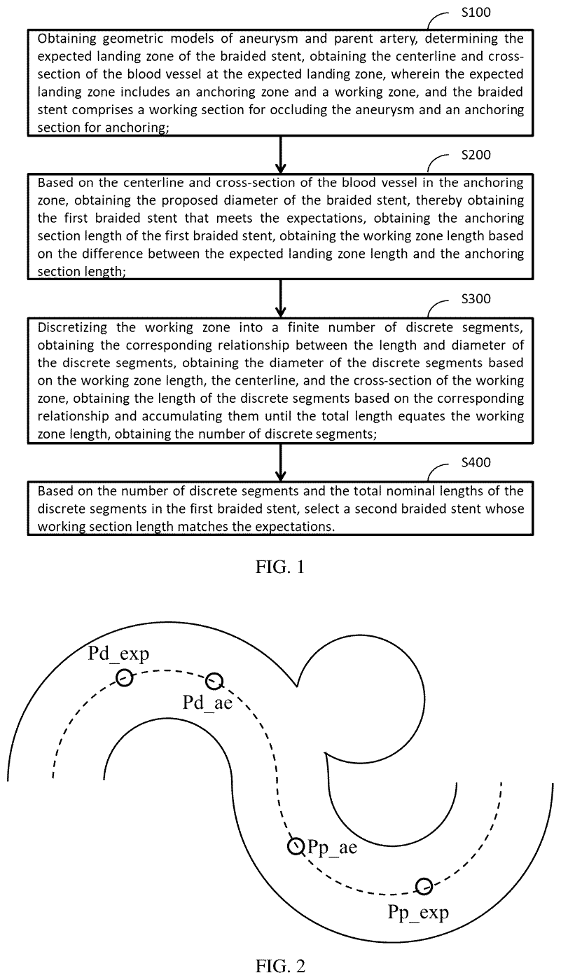

In order to solve the above technical problems, referring to FIG. 1-FIG. 4, a sizing method for braided stents (referred to as stent) is provided in an embodiment of the present application, including:
Step S100, Obtaining geometric models of aneurysm and parent artery, determining the expected landing zone of the braided stent, obtaining the centerline and cross-section of the blood vessel at the expected landing zone, wherein the expected landing zone includes an anchoring zones and a working zone, and the braided stent comprises a working section for occluding the aneurysm and an anchoring section for anchoring;
Step S200, Based on the centerline and cross-section of the blood vessel in the anchoring zone, obtaining the proposed diameter of the braided stent, thereby obtaining the first braided stent that meets the expectations, obtaining the anchoring section length of the first braided stent, obtaining the working zone length based on the difference between the expected landing zone length and the anchoring section length;
Step S300, Discretizing the working zone into a finite number of discrete segments, obtaining the corresponding relationship between the length and diameter of the discrete segments, obtaining the diameter of the discrete segments based on the working zone length, the centerline, and the cross-section of the working zone, obtaining the length of the discrete segments based on the corresponding relationship and accumulating them until the total length equates the working zone length, obtaining the number of discrete segments;
Step S400, Based on the number of discrete segments and the total nominal lengths of the discrete segments in the first braided stent, select a second braided stent whose working section length matches the expectations.

The expected landing zone refers to the location of the vascular channel where the braided stent needs to be implanted, including the aneurysm and the parent artery. Generally, it is determined based on the radiological images of the patient. The working zone is the zone between Pd_ac and Pp_ac as shown in FIG. 2. The anchoring section is used for implantation in the anchoring zone, while the working section is used for implantation in the working zone. Due to objective conditions, it may not be possible to find an exact match for the anchoring zone and working zone for the patient. Therefore, the anchoring zone and section, and the working zone and section, is not strictly limited to being completely consistent. In other words, at least a portion of the working section of the braided stent is placed in the working zone during implantation, and at least a portion of the anchoring section is placed in the anchoring zone. Specifically, during use, the anchoring section apposes to the inner wall of the blood vessel by its own elastic force to provide support, while the working section occludes the aneurysm neck. The combination of the two achieves embolism of the aneurysm. In this embodiment, the first braided stent and the second braided stent refer to the braided stents that meet their respective criteria. The actual range of their numbers is determined based on the range of criteria they meet.

The anchoring section of this embodiment is used to obtain the proposed diameter of the braided stent, while the working section is used to obtain the proposed length of the braided stent's working section. The proposed diameter is obtained based on the size of the vessel lumen in the anchoring zone. By simulating the implantation status of discrete segments into the working zone, the number of discrete segments required to fill the working zone length under the proposed diameter is obtained, and then a second braided stent that meets the requirement of the working zone length is selected. Through the proposal based on these two dimensions, a braided stent suitable for the patient to be implanted is finally obtained, providing reference basis for the selection of the braided stent and assisting doctors in accurate and efficient planning of the stent model before or during surgery.

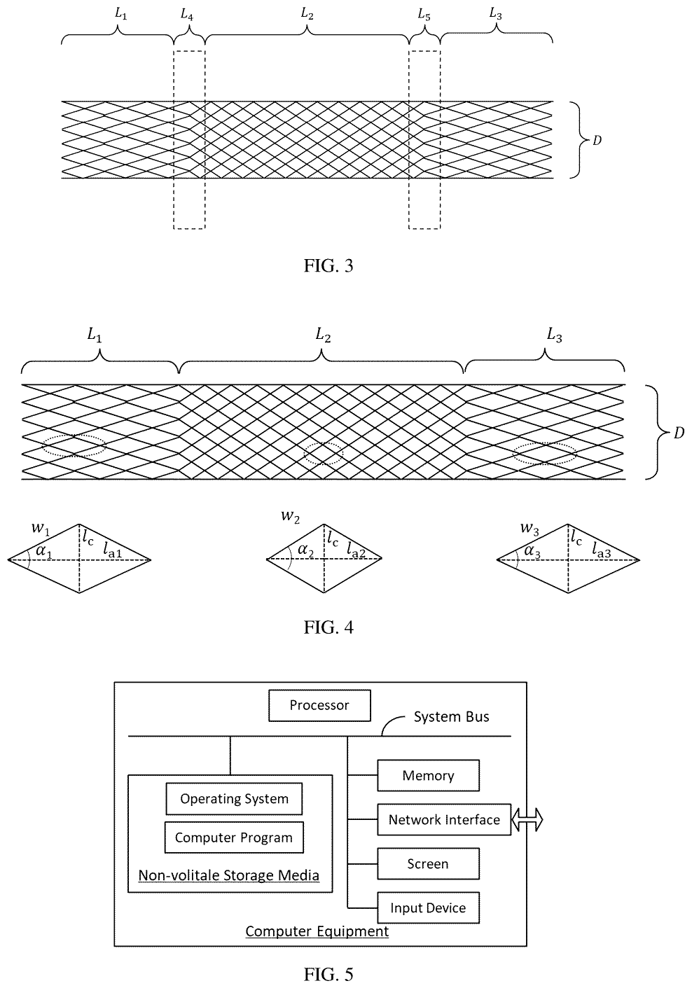

Referring to FIGS. 3 and 4, considering the implantation purpose of the braided stent is to occlude the area where the aneurysm is located and reduce occlusion in non-aneurysmal areas. Furthermore, the braided stent is a non-uniform braided stent, where the anchoring section comprises two parts located at the opposite ends of the working section, namely the first part l₁ and the third part l₃, while the working section (second part l₂) has a higher braiding density than the anchoring section. The anchoring section of the non-uniform braided stent is relatively sparse, and any length changes that occur during its implantation can be ignored. Between the anchoring section and the working section, there may also be a transition zone, the length of which can be considered as zero, resulting in the structural schematic shown in FIG. 4.

Referring to FIG. 2, in step S200, the proposed diameter of the braided stent is obtained based on the centerline and cross-section of the vessel in the anchoring zone. Specifically, the anchoring zone includes the distal anchoring zone (between Pd_exp and Pd_ac) and the proximal anchoring zone (between Pp_exp and Pp_ac), with the working zone located between Pd_ac and Pp_ac. The distal anchoring zone has the distal anchoring point Pd_exp, relatively distant from the aneurysm, while the proximal anchoring zone has the proximal anchoring point Pp_exp, also relatively distant from the aneurysm. The larger of the radius between the distal and proximal anchoring points is chosen as the reference diameter, and the proposed diameter is obtained based on this reference diameter. The proposed diameter obtained based on the reference diameter can be determined by those skilled in the field, generally slightly larger than the reference diameter to ensure the apposition of the anchoring section.

Referring to FIG. 4, in step S300, discretizing the working zone into a finite number of discrete segments and obtaining the corresponding relationship between the length and diameter of the discrete segments, specifically comprising: The repeating unit cell of the working section is a rhombus formed by the intersection of the stent wires, with the length of the rhombus along the axial direction of the braided stent being the length of the discrete segment. Based on the relationship between the length and height of the rhombus and the circumferential arrangement of the rhombi, obtain the corresponding relationship between the length and diameter of the discrete segments.

Discretizing the working zone into a finite number of discrete segments and obtaining the corresponding relationship between the length and diameter of the discrete segments, utilizing the following equation:

$${l}_{a 2} ( D ) = 2 \sqrt{{w}_{2}^{2} - \frac{{π}^{2} {( D - d )}^{2}}{{N}^{2}}}$$

In the equation, lₐ₂ (D) represents the length of the discrete segment, D represents the diameter of the discrete segment;
w₂ represents the rhombus side length, N represents the number of stent wires, d represents the diameter of the stent wires, all of which are parameters of the first braided stent.

In step S300, obtaining the diameter of the discrete segments based on the length of the working zone, the centerline, and the cross-section of the working zone, utilizing the following equation:

$${R}^{*} ( s ) = min ( R ( s ) , \frac{1}{2} {D}_{free} )$$

In the equation, s represents a point on the blood vessel centerline, R(s) represents the radius of the blood vessel centerline, R*(s) represents the radius of the discrete segment which is used to obtain the diameter of the discrete segment, and D_{free} represents the upper limit of the expanded diameter of the first braided stent in its natural released state.

In one embodiment, a sizing method for braided stent is provided, corresponding to steps S100 to S400 as described above. It includes: (1) Stent segmentation; (2) Determination of mesh size and quantity for different stent sections; (3) Establishment of local foreshortening model; (4) Image reading and surface reconstruction; (5) Extraction of region of interest; (6) Generation of vascular centerline and calculation of parameters along the centerline; (7) Determination of diameter specification; (8) Determination of working zone; (9) Determination of length of stent working section.

(1) Stent segmentation, corresponding to the working section and anchoring section obtained in step S100.

Firstly, the geometric structure of the non-uniform braided stent is decomposed along the axial direction. As shown in FIG. 3, the geometric structure of the non-uniform braided stent can be divided into 5 parts: the central working part, two anchoring parts at both ends, and the transition parts between the working part and the anchoring parts (inside the dashed rectangle in FIG. 1). The 5 parts of the stent are labeled as the first part, second part, third part, fourth part, and fifth part, with lengths labeled as l₁, l₂, l₃, l₄, l₅, respectively.

The manufacturing process typically determines that the working section of the non-uniform braided stent continuously transitions to the anchoring section, and there is no clear boundary between the transition part and the anchoring section. Manufacturers usually do not explicitly define the length of the transition part, so the length of the transition part can be simply set to 0. In this case, the simplified geometric structure of the non-uniform braided stent is shown in FIG. 4.

If the manufacturer specifies the length of the transition part or even provides a distribution function for the mesh size of the transition part, this technique can still accurately simulate the stent by further subdividing the transition part, without affecting the implementation of the technical solutions of various embodiments of the present application. Even if a uniform braided stent is used, it can still be divided into anchoring section and working section based on function, and the specific position of implantation of the anchoring section can be adjusted according to its mechanical structural adaptability. In this case, the lengths of the first and third part are both equal to 0, and the non-uniform braided stent degenerates into a uniform braided stent.

(2) Determination of mesh size and quantity for different stent sections.

The geometric structure of the braided stents can be described by representative diamond-shaped pore, and the foreshortening behavior of the braided stents is also determined by the geometric characteristics of the representative pore. Each representative pore contains axial diagonal length lₐ, circumferential diagonal length l_{c}, side length w, and braiding angle α. Since the number of stent wires remains constant for different sections of the stents, l_{c} remains constant for different parts, while other parameters vary, as shown in FIG. 4.

Utilize the following formula to determine the number of pores along the axial direction of the stent for each part:

$${n}_{i} = {L}_{i} / {l}_{a i} , i = 1 , 2 , 3$$

(3) Establishment of local foreshortening model corresponds to obtaining the relationship between the length and diameter of the discrete segments.

The local foreshortening model of the stent is used to describe the correspondence between the axial length of the pore and the stent diameter in each part of the stent.

After the stent is released in the blood vessel, its cross-section keeps circular. It is assumed that the side length wᵢ of the stent pore remain unchanged, meaning the overlapping positions of the stent wires can only undergo relative rotation but no sliding.

Based on these two fundamental assumptions, the one-to-one correspondence between the stent diameter D and the axial diagonal length lₐᵢ of the mesh can be obtained as follows:

$${l}_{a i} ( D ) = 2 \sqrt{{w}_{i}^{2} - \frac{{{π}^{2} ( D - d )}^{2}}{{N}^{2}}} , i = 1 , 2 , 3$$

Where N is the number of stent wires, D is the diameter of the stent, and d is the diameter of the stent wire.

Since the length of the anchoring section of the non-uniformly braided stent remains basically unchanged after implantation in the blood vessel, the foreshortening of the anchoring section is negligible. This local foreshortening model is mainly used to describe the correspondence between the discrete segment length and the discrete segment diameter, i.e., the case when i=2.

(4)˜ (6) correspond to obtaining the centerline and cross-section of the blood vessel at the expected landing zone and the working zone and anchoring zone included.

(4) Image Reading and Surface Reconstruction, including: Reading vascular images, including but not limited to three-dimensional image sequences of DSA, CTA, and MRA. Utilizing thresholding, level set method, or artificial intelligence segmentation models (such as 3D UNet) to segment the image sequences, then using marching cubes algorithm to reconstruct the surface, obtaining the vascular model.

(5) Extraction of region of Interest (ROI), including: Extracting the regions of interest from the vascular model, retaining the models of the aneurysm and the parent artery. Specific interactive methods may involve selecting vascular structures within a transparent clipping sphere through zooming and panning, or manually clipping off unnecessary vascular branches one by one.

(6) Generation of vascular centerline and calculation of parameters along the centerline, including: Calculating the Voronoi diagram from the proximal inlet of the vessel to each distal outlet. Based on each Voronoi diagram, obtaining the sequence of coordinates of the centerline points starting from the proximal inlet and ending at each distal outlet, along with the corresponding along-line radii (maximum inscribed sphere radii) sequence. Calculating the tangent unit vector, principal normal vector, and secondary normal vector at each point along the centerline based on the sequence of coordinates of the centerline points. Calculating the curvature radius at each point along the centerline, as well as the vascular cross-sectional area and circumference at each point. At this point, obtaining the structural model containing the aneurysm and the arteries carrying the aneurysm, as well as the vascular centerline and vascular cross-sections at the expected landing zone.

(7) Determination of diameter specification, corresponding to step S200, involves obtaining the proposed diameter of the braided stent based on the vascular centerline and cross-section in the anchoring zone. Subsequently, obtaining the first braided stent that meets expectations and obtaining the anchoring section length of the first braided stent.

Manually selecting the expected distal anchoring point Pd_exp and proximal anchoring point Pp_exp on the target centerline to obtain the along-line radii at the two anchoring point positions and comparing their sizes. The larger value is taken as the reference radius, and twice the reference radius is taken as the reference diameter. Filtering all diameter specifications in the stent's diameter specification library that are greater than the reference diameter, selecting the minimum value as the proposed diameter specification. Based on the proposed diameter specifications of the first braided stent, determining the lengths of the distal anchoring part L₁ and the proximal anchoring part L₃.

(8) Determination of working zone, corresponding to step S200, involves obtaining the length of the working zone based on the difference between the length of the expected landing zone and the anchoring zone length.

Starting from the expected distal anchoring point Pd_exp, search along the centerline towards the proximal direction to find the end point of the distal anchoring zone Pd_ac. The along-line length between Pd_exp and Pd_ae equals L₁. Starting from the expected proximal anchoring point Pp_exp, search along the centerline towards the distal direction to find the end point of the proximal anchoring zone Pp_ac. The along-line length between Pp_exp and Pp_ac equals L₃.

Thus, subtracting the anchoring zone lengths from the expected landing zone yields the length of the working zone. The central part between Pd_ac and Pp_ac along the centerline constitutes the coverage area of the stent working section after implantation. Let the length of the centerline between Pd_ac and Pp_ac be denoted as L_{w}, which is the length of the working zone.

(9) Determination of length of stent working section, corresponding to step S300, involves obtaining the discrete segment diameters based on the length of the working zone, the vascular centerline, and cross-section within the working zone. Then, obtaining the discrete segment lengths based on the corresponding relationship and accumulating them until the total length equates the length of the working zone, thus determining the number of discrete segments.

Since the maximum deployment diameter of the stent in its natural released state (without additional intraoperative maneuvers) is D_{free}, the initial processing of the along-line radii needs to be performed.

$${R}^{*} ( s ) = min ( R ( s ) , \frac{1}{2} {D}_{free} )$$

Where represents a point on the centerline, is the initial along-line radius, which is the along-line radius of the vascular centerline, and is the processed along-line radius, which represents the radius of the discrete segment.

Furthermore, the discrete segments include sequentially determined first and second discrete segments. In step S300, the lengths of the discrete segments are obtained based on the corresponding relationship, specifically including: determining the starting point of the first discrete segment in the working zone, and obtaining the length of the first discrete segment based on the corresponding relationship; using the endpoint of the first discrete segment as the starting point for the second discrete segment, and obtaining the length of the second discrete segment based on the corresponding relationship.

In step S300, the number of discrete segments is obtained by accumulating the lengths of all discrete segments until the total length equates the length of the working zone. Specifically, when the lengths of all discrete segments accumulate to the length of the working zone, the accumulated number of discrete segments is obtained. In this process, subsequent discrete segments, such as the third discrete segment, fourth discrete segment, and so on, are executed sequentially. Once the length of the entire working zone is filled using discrete segments, the operation is stopped.

Specifically, let Pd_ac be denoted as P. Starting from P, using the foreshortening model of the working section (the second part), the deployed length of the first pore lₐ₂ (2R*(s_{P})) is calculated, and a new point P_new is searched towards Pp_ac based on this length. The distance from point P_new to P equals lₐ₂(2R*(s_{P})). Subsequently, the three-dimensional coordinates, along-line radius, and other along-line parameters of the position P_new are obtained, and P_new is set as P. This process is repeated, while accumulating the deployed lengths of the pores and counting the pores until the total length is greater than or equal to L_{w}. This determines the number of pores, which is equivalent to the accumulated number of discrete segments.

At this point, by multiplying the number of pores n by the axial length lₐ₂ of the pores in the nominal state of the proposed first braided stent, the expected length of the stent working section L₂ is obtained. However, since the length of the working section of the second selected braided stent may not be exactly identical to the expected value, the braided stent with a working section length closest to L₂ is selected as the proposed second braided stent.

The second braided stent itself comprises the first part, the second part, and the third part, with the lengths of all three parts already known. Upon determining the length specifications of the stent, accordingly, the number of along-line pores n₁, n₂, and n₃ in the three parts of the stent can be determined.

In one embodiment, the sizing method also includes simulating the implantation of the working section of the second braided stent to assess its implantation results, including:

Step S500: Obtain the length of the working section of the second braided stent, discretize the length of the working section of the second braided stent into a finite number of discrete segments, and based on the expected landing zone, the vascular centerline at the expected landing zone, and the vascular cross-section, obtain the diameter of each discrete segment.

Step S600: Based on the diameter of the discrete segments and corresponding relationship, obtain the length of each discrete segment. Simulate the implantation of the working section of the second braided stent in the working zone until the implantation of all discrete segments of the second braided stent is completed, obtaining the endpoint of the working section of the second braided stent after implantation.

Specifically, let Pd_exp be denoted as P. Starting from P, the deployed length lₐ₁(2R*(s_{P})) of the first pore is calculated. If a non-uniform braided stent is adopted, lₐ₁(2R*(s_{P})) is a constant determined by the specifications of the second braided stent. Based on this length lₐ₁(2R*(s_{P})), a new point P_new is searched towards the proximal end of the centerline. The along-line distance from P_new to P equals lₐ₁(2R*(s_{P})).

Next, obtain the three-dimensional coordinates, along-line radius, and other along-line parameters of the position P_new, and set P_new as P. Repeat the above steps and count the pores until the number of pores equals n₁. Then, start from the latest point P, utilize the foreshortening model of the second part to sequentially calculate the deployed length lₐ₂(2R*(s_{P})) of the pores in the second part, and count the number of pores until the number of pores equals n₂. Furthermore, repeat the above steps for the third part until the number of pores equals n₃. Similarly, if a non-uniform braided stent is adopted, the length of the third part is a constant determined by the specifications of the second braided stent.

The sizing method provided in each embodiment can propose the diameter and length of non-uniform braided stents in real time based on the user's expected landing zone and conduct virtual implantation, with accurate results. This method can lower the clinical usage threshold of braided stents, reduce the difficulty for surgeons, alleviate pressure, and improve surgical outcomes.

It should be understood that although the steps in the flowchart of FIG. 1 are shown sequentially according to the direction of the arrows, these steps are not necessarily executed strictly in the order indicated by the arrows. Unless otherwise specified in this document, there is no strict order limitation on the execution of these steps, and these steps can be performed in a different order. Additionally, at least some steps in FIG. 1 may include multiple sub-steps or stages, which may not necessarily be completed simultaneously but can be executed at different time. The execution order of these sub-steps or stages is not necessarily sequential, but can alternate or be interleaved with at least some sub-steps or stages of other steps.

In one embodiment, a set of computer equipment is provided, which can be a terminal, with an internal structure diagram shown in FIG. 5. The computer device includes a processor, memory, network interface, display screen, and input device connected via a system bus. The processor of the computer device provides computing and control capabilities. The memory of the computer device includes non-volatile storage media and volatile memory. The non-volatile storage media stores the operating system and computer programs, while the volatile memory provides an environment for running the operating system and computer programs stored in the non-volatile storage media. The network interface of the computer device is used for communication with external terminals via network connections. The computer program executed by the processor realizes a model recommendation method for braided stents. The display screen of the computer device can be a liquid crystal display screen or an electronic ink display screen, and the input device of the computer device can be a touch layer overlaid on the display screen, buttons, trackballs, or touchpads set on the outer shell of the computer device, as well as external keyboards, touchpads, or mice.

In another embodiment, a computer device is provided, comprising memory and a processor, where the memory stores a computer program, and the processor executes the computer program to implement the following steps:
Step S100, Obtaining geometric models of aneurysm and parent artery, determining the expected landing zone of the braided stent, obtaining the centerline and cross-section of the blood vessel at the expected landing zone, wherein the expected landing zone includes an anchoring zones and a working zone, and the braided stent comprises a working section for occluding the aneurysm and an anchoring section for anchoring;
Step S200, Based on the centerline and cross-section of the blood vessel in the anchoring zone, obtaining the proposed diameter of the braided stent, thereby obtaining the first braided stent that meets the expectations, obtaining the anchoring section length of the first braided stent, obtaining the working zone length based on the difference between the expected landing zone length and the anchoring section length;
Step S300, Discretizing the working zone into a finite number of discrete segments, obtaining the corresponding relationship between the length and diameter of the discrete segments, obtaining the diameter of the discrete segments based on the working zone length, the centerline, and the cross-section of the working zone, obtaining the length of the discrete segments based on the corresponding relationship and accumulating them until the total length equates the working zone length, obtaining the number of discrete segments;
Step S400, Based on the number of discrete segments and the total nominal lengths of the discrete segments in the first braided stent, select a second braided stent whose working section length matches the expectations.

Those skilled in the field will understand that all or part of the processes in the above embodiments can be completed by instructing the relevant hardware through a computer program. The computer program may be stored in a non-volatile computer-readable storage medium, and when executed by the processor, may include the processes of the embodiments described above. Furthermore, any reference to memory, storage, database, or other media in the embodiments provided herein may include both non-volatile and/or volatile storage media. Non-volatile storage media may include read-only memory (ROM), programmable ROM (PROM), erasable programmable ROM (EPROM), electrically erasable programmable ROM (EEPROM), or flash memory. Volatile storage media may include random access memory (RAM) or external cache memory. As an illustration and not limitation, RAM may be obtained in various forms such as static RAM (SRAM), dynamic RAM (DRAM), synchronous DRAM (SDRAM), double data rate SDRAM (DDR SDRAM), enhanced SDRAM (ESDRAM), synchronous link (Synchlink) DRAM (SLDRAM), Rambus direct RAM (RDRAM), direct dynamic RAM (DRDRAM), and Rambus DRAM (RDRAM).

Various technical features of the above embodiments can be combined arbitrarily. For the sake of brevity, not all possible combinations of technical features in the above embodiments are described. However, as long as the combination of these technical features does not conflict, it should be considered within the scope of this specification. When technical features of different embodiments are reflected in the same drawing, it can be considered that the drawing also simultaneously discloses combinations of the embodiments involved.

The above-described embodiments only express several embodiments of the present application, which are specific and detailed, but should not be understood as limiting the scope of the disclosure. It should be noted that those skilled in the field can make various modifications and improvements without departing from the concept of the present application. These modifications and improvements are within the scope of protection of the present application. Therefore, the scope of protection of the patent application should be determined based on the appended claims.

1. The sizing method for braided stents is characterized in that, comprising:
obtaining geometric models of aneurysm and parent artery, determining the expected landing zone of the braided stent, obtaining the centerline and cross-section of the blood vessel at the expected landing zone, wherein the expected landing zone comprises an anchoring zones and a working zone, and the braided stent comprises a working section for occluding the aneurysm and an anchoring section for anchoring;
based on the centerline and cross-section of the blood vessel in the anchoring zone, obtaining the proposed diameter of the braided stent, thereby obtaining the first braided stent that meets the expectations, obtaining the anchoring section length of the first braided stent, obtaining the working zone length based on the difference between the expected landing zone length and the anchoring section length;
discretizing the working zone into a finite number of discrete segments, obtaining the corresponding relationship between the length and diameter of the discrete segments, obtaining the diameter of the discrete segments based on the working zone length, the centerline, and the cross-section of the working zone, obtaining the length of the discrete segments based on the corresponding relationship and accumulating them until the total length equates the working zone length, obtaining the number of discrete segments; and
based on the number of discrete segments and the total nominal lengths of the discrete segments in the first braided stent, select a second braided stent whose working section length matches the expectations.

2. The sizing method for braided stents according to claim 1, wherein the braided stent is a non-uniform braided stent, and wherein the anchoring section comprises two parts located at two opposite ends of the working section, and the braiding density of the working section is higher than that of the anchoring section.

3. The sizing method for braided stents according to claim 2, wherein obtaining the proposed diameter of the braided stent based on the centerline and cross-section of the anchoring zone, specifically comprises:
the anchoring zone comprises a distal anchoring zone and a proximal anchoring zone, wherein the distal anchoring zone has a distal anchoring point relatively far from the aneurysm, and the proximal anchoring zone has a proximal anchoring point relatively far from the aneurysm. Selecting the larger radius of the two, either the distal anchoring point or the proximal anchoring point of the blood vessel centerline as the half of reference diameter, and obtaining the proposed diameter based on the reference diameter.

4. The sizing method for braided stents according to claim 1, wherein discretizing the working zone into a finite number of discrete segments and obtaining the corresponding relationship between the length and diameter of the discrete segments, specifically comprises:
the repeating unit cell of the working section is a rhombus formed by the intersection of the stent wires, with the length of the rhombus along the axial direction of the braided stent being the length of the discrete segment. Based on the relationship between the length and height of the rhombus and the circumferential arrangement of the rhombi, obtain the corresponding relationship between the length and diameter of the discrete segments.

5. The sizing method for braided stents according to claim 4, wherein discretizing the working zone into a finite number of discrete segments and obtaining the corresponding relationship between the length and diameter of the discrete segments, utilize the following equation:
$${l}_{a 2} ( D ) = 22 \sqrt{{w}_{2}^{2} - \frac{{π}^{2} {( D - d )}^{2}}{{N}^{2}}}$$
in the equation, lₐ₂(D) represents the length of the discrete segment, D represents the diameter of the discrete segment;
w₂ represents the rhombus side length, N represents the number of stent wires, d represents the diameter of the stent wires, all of which are parameters of the first braided stent.

6. The sizing method for braided stents according to claim 1, wherein obtaining the diameter of the discrete segments based on the length of the working zone, the centerline, and the cross-section of the working zone, utilize the following equation:
$${R}^{*} ( s ) = min ( R ( s ) , \frac{1}{2} {D}_{free} )$$
in the equation, s represents a point on the blood vessel centerline, R(s) represents the radius of the blood vessel centerline, R*(s) represents the radius of the discrete segment which is used to obtain the diameter of the discrete segment, and D_{free} represents the upper limit of the expanded diameter of the first braided stent in its natural released state.

7. The sizing method for braided stents according to claim 1 wherein the discrete segments comprises sequentially determined first and second discrete segments;
and wherein obtaining the length of the discrete segments based on the corresponding relationship, specifically comprises:
determining the starting point of the first discrete segment in the working zone, obtaining the length of the first discrete segment based on the corresponding relationship; and
using the endpoint of the first discrete segment as the starting point of the second discrete segment, obtaining the length of the second discrete segment based on the corresponding relationship.

8. The sizing method for braided stents according to claim 7, wherein obtaining the number of discrete segments until the total length equates to the length of the working zone, specifically comprises:
obtaining the accumulated number of discrete segments when the total length of the discrete segments equates to the length of the working zone.

9. The sizing method for braided stents according to claim 7, further
comprising: simulating the implantation of the working section of the second braided stent:
obtaining the length of the working section of the second braided stent, discretizing the length of the working section of the second braided stent into a finite number of discrete segments, obtaining the diameter of the discrete segments based on the expected landing zone, the centerline, and the cross-section of the blood vessel at the expected landing zone; and
obtaining the length of the discrete segments based on the diameter of the discrete segment and corresponding relationship, simulating the implantation of the working section of the second braided stent into the working zone, until the simulation of implantation of all discrete segments of the second braided stent is completed, and obtaining the endpoint of the working section of the second braided stent after implantation.

10. The computer equipment comprises a memory, a processor, and a computer program stored in the memory, wherein the processor executes the computer program to implement the steps of the sizing method for braided stents according to claim 1.
